(19) United States
(12) Patent Application Publication
(10) Pub. No.: US 2021/0353421 A1
(43) Pub. Date: Nov. 18, 2021

(54) VENOUS VALVE PROSTHESIS

(71) Applicant: Renovo Medsolutions, LLC, Redwood City, CA (US)

(72) Inventor: Lee Bolduc, Redwood City, CA (US)

(73) Assignee: Renovo Medsolutions, LLC, Redwood City, CA (US)

(21) Appl. No.: 17/364,550

(22) Filed: Jun. 30, 2021

Related U.S. Application Data
(63) Continuation of application No. PCT/US19/68502, filed on Dec. 24, 2019.
(60) Provisional application No. 62/788,055, filed on Jan. 3, 2019.

(51) Int. Cl. A61F 2/24

(52) U.S. Cl. CPC A61F 2/2475; A61F 2250/001; A61F 2/2418

(57) ABSTRACT

An implantable prosthetic valve includes an outer tubular wall or other body having an axial passage extending from an inlet end to an outlet end. The wall may be constructed of a flexible biocompatible material, and a pair of apposed valve leaflets is usually disposed in the axial passageway of the outer tubular wall or body. Each valve leaflet has an inlet edge, an outlet edge, and a pair of lateral edges extending between the inlet edge and the outlet edge. The outlet edges are configured to be normally open to allow unrestricted fluid flow and close together when fluid pressure is applied to the outlet end. In some instances, the inlet edge, the outlet edge, and the pair of lateral edges of each leaflet are integrated along their entire lengths into the outer tubular wall. In other instances, the leaflets may be formed as a separate structure or assembly that is attached within the tubular wall or body. In still other instances, each leaflet may be recessed along its length in a direction toward the inlet end.

### CROSS-REFERENCE TO RELATED APPLICATIONS

This application is a continuation of PCT Application No. PCT/US19/68502 (Attorney Docket No. 51203-704.601), filed Dec. 24, 2019, which claims the benefit of U.S. Provisional No. 62/788,055, (Attorney Docket No. 51203-704.101), filed Jan. 3, 2019, the entire content of which is incorporated herein.

### BACKGROUND OF THE INVENTION

### 1. Field of the Invention

The present invention relates generally to the field of implantable prosthetic vascular valves and more specifically to implantable prosthetic venous valves designed to replace diseased, damaged or clinically incompetent valves in the human vascular system.

The peripheral venous system in the human body functions as a reserve to store blood and as a conduit to return blood to the heart. The lower extremities within the human venous system contain a number of one-way bicuspid valves that allow forward (antegrade) blood flow to the heart while preventing reverse (retrograde) blood flow to the feet. Lower limb muscular contraction allows the body to overcome the force of gravity and pump blood back to the heart. The one-way bicuspid valves (venous valves) facilitate this pumping action by preventing the blood from draining back to the feet and/or pooling in the lower extremities. However, patients with diseased or damaged (incompetent) venous valves can develop Chronic Venous Insufficiency (CVI), which is characterized by chronic venous hypertension from blood pooling in the lower limbs. Venous hypertension and CVI can result in chronic leg pain, varicose veins, fatigue, venous edema, skin inflammation, induration, and ulceration.

While CVI is not life-threatening, the condition can be extremely painful and disabling. The socio-economic impact of symptomatic CVI is significant, imposing financial burden and resulting in impaired ability to engage in social and occupational activities. It has been estimated that approximately 2.5 million people in the United States have CVI and of those, approximately 20% develop venous ulcers¹, which represents the most severe manifestation of CVI and is the highest level of CEAP (Clinical, Etiology, Anatomic, Pathophysiology classification scheme for reporting, diagnosing and treating CVI).

The specific treatment for CVI is based on the severity of disease. Current treatments for CVI include compression, medication, vein ablation, venous stenting, vein bypass, and vein valve reconstruction/replacement.

The effectiveness of the treatments to reduce the symptom severity of CVI has demonstrated varying levels of success. In the most severe cases, surgical intervention is required when the response to conservative measures are unsatisfactory at relieving the symptoms of CVI. Venous valve reconstruction, or valvuloplasty, can be performed as an open surgical procedure and as a less invasive closed procedure. Venous valvuloplasty has been shown to provide 59% competency and 63% ulcer-free recurrence at 30 months. Complications from the venous valvuloplasty include bleeding (because patients need to remain anticoagulated), deep vein thrombosis (DVT), pulmonary embolism, ulcer reoccurrence, and wound infections¹. Because of the complications and limited success rate, surgical venous valve reconstruction is not routinely performed and is only considered in selected patients.

Currently, there are no commercially available prosthetic venous valves designed to replace diseased or damaged natural venous valves. Several attempts at developing prosthetic venous valves have been made since the introduction of vascular valve implants. These attempts most often rely upon techniques and materials that have been used successfully in cardiac valve replacements. The challenges associated with the venous system include a wide range of pressures and flow rates with a higher risk of thrombosis due to blood stagnation and/or higher shear rates. Such complications have prevented these technologies from producing a successful venous valve replacement.

Accordingly, there is a need for a prosthetic venous valve, implantable through a less invasive procedure, to replace a damaged or diseased natural valve. It would be particularly beneficial if such prosthetic venous valves are fabricated in whole or in part from materials which can withstand the challenging venous environment in order to provide a long-lasting solution for CVI.

### 2. Listing of the Background Art

Relevant patents and patent publications include Ku et al. US2016/0256277; Long et al. US2008/0091261; Ku et al. US20120053676; Edelman et al. U.S. Pat. No. 9,056,006; Sathe et al. US2008/0269879; Acosta et al. U.S. Pat. No. 8,246,676; Paul, Jr. et al. U.S. Pat. No. 8,679,175; Shoemaker et al. U.S. Pat. No. 8,721,717; Shoemaker et al. U.S. Pat. No. 8,128,681; Hill et al. U.S. Pat. No. 8,012,198; Hill et al. U.S. Pat. No. 7,867,274; Acosta et al. U.S. Pat. No. 6,958,076; Duerig et al. U.S. Pat. No. 6,503,272; Greenhalgh U.S. Pat. No. 6,494,909; Kirk et al. US2017/0196692; Quintessenza US2013/0310927; Kelly US2013/0304196; Wilder et al. US2003/0171802; Fearnot et al. U.S. Pat. No. 8,038,710; Sarac et al. U.S. Pat. No. 7,547,322; and Sweeney et al. US2014/0257463. See also, Padala et al. (2009) Ann. Thorac. Surg. 88:1499-1504.

### SUMMARY OF THE INVENTION

In the first aspect of the present invention, a prosthesis that is implantable through a less invasive procedure includes an outer tubular body and valve which permit blood flow in one direction and prevents blood flow in the reverse direction. The outer tubular body is typically cylindrical in shape and can extend over the entire length, a portion of the length, or extend beyond the entire length of the prosthesis.

The outer tubular body is typically elastic or otherwise deformable to accommodate the shape of the blood vessel or other lumen into which it is implanted. Typically, the outer tubular body will incorporate reinforcement or other structural element(s) to provide radial support and stability to the prosthesis. The reinforcement or other structural element(s) can be polymeric, metallic, ceramic, and combinations thereof, and can fully or partially enclose the outer tubular body, be embedded wholly or partially within the wall of the outer tubular body, or fully or partially line the outer tubular body. In exemplary embodiments, the reinforcement or other structural element(s) will be embedded wholly or partially within the wall of the outer tubular body.

The reinforcement or other structural element(s) can extend over a portion of the prosthesis length or over the entire length. In a preferred embodiment, the reinforcement or other structural element(s) will contain interstitial spaces that allow the reinforcement or other structural element(s) to be deformed to facilitate access to a target location within the body for implantation. In addition, the interstitial spaces can allow a base or matrix material of the prosthesis to interact and/or connect the reinforcement or other structural element(s) to the base or matrix material. This connection of the reinforcement or other structural element(s) to the base or matrix material can be accomplished through chemical or mechanical means, such as adhesives, physical attachments or material encapsulation, such as through molding. The reinforcement or other structural element(s) could be designed to self-expand after delivery to a target location within a human body, or alternately, it could be expanded through mechanical means, such as a balloon or a mechanical expander.

In a preferred embodiment, the prosthesis base or matrix material will be in blood contact when implanted in the target location within the human body and the reinforcement or other structural element(s) will have limited blood contact. In one embodiment, the reinforcement or other structural element(s) will be completely encapsulated and/or covered by the base or matrix material. In an alternative embodiment, the reinforcement or other structural element(s) will have specific features and/or areas that are devoid of the base or matrix material.

The reinforcement or other structural element(s) may also incorporate features to provide stability and/or migration resistance of the prosthesis during or after implantation. Suitable features include barbs, hooks, and other tissue anchors. Alternatively or additionally, the external surface of the outer tubular body may be roughened or textured in a region where the prosthesis is in contact with the native tissue to promote tissue adhesion or in-growth. These features and/or texturing could be circumferentially and/or axially arrayed or distributed over the exterior surface of the outer tubular body. Additionally, the reinforcement or other structural element(s) may also include features that facilitate the connection to the base or matrix material.

The base or matrix material of the prosthesis, including at least the outer tubular body and valve leaflets, will be biocompatible, non-thrombogenic and have a suitable flexibility and durability for use as a vascular implant. Suitable materials of the present invention for fabrication of at least the outer tubular body and valve leaflets include but are not limited to, polyesters, polyethylenes, fluoropolymers (such as ePTFE), silicones, and hydrogels (such as polyvinyl alcohols (PVA)).

In a preferred embodiment, the tubular body and leaflets of the prosthesis are made from a single, homogeneous material (referred to herein as an “integrated structure”); however, it is contemplated that the type, structure and properties of the base or matrix material could vary in different regions within the prosthesis to improve or alter flexibility, durability, and/or strength. In addition, the base or matrix material may be a mixture or composite of two or more materials selected to achieve the desired flexibility, durability, and/or strength. Exemplary additional materials include, but are not limited to, filaments, strands, nanorods, and the like, which may be provided to provide reinforcement as described above. The base or matrix material may also include radiopaque material that allows visualization of the location and orientation of the prosthesis during and/or after the process of implantation within the human body. Alternatively, the radiopaque material (markers) could be integral or attached to the reinforcement or other structural element(s) for the purposes of visualization of the prosthesis during and/or after the process of implantation.

The prostheses of the present invention will include two or more valve leaflets, where the leaflets are typically formed in a “normally open” configuration (i.e. open in their unstressed or “shelf” condition) and adapted to allow flow in one direction while closing in response to flow in a reverse direction. Inlet ends of the leaflets will typically be aligned with or adjacent to the inlet of the outer tubular body of the prosthesis.

In exemplary embodiments, the prosthesis will have two leaflets and the inlet will be generally circular in shape (when unstressed). The wall thickness of each leaflets can be uniform or non-uniform, for example being thicker near an inlet end (where the leaflet is attached to an inner wall of the outer tubular body) and thinner near an outlet end (where the leaflet is unattached and free to open and close). Additionally or alternatively, the thickness of leaflet can be uniform or vary across its length and or width (generally along a longitudinal and or major axis of the outer tubular body as defined with respect to the axes shown in FIG. 1A), for example being thicker in the middle of the leaflet and thinner near the inner wall attachment locations.

The length of each leaflet will typically be from 75% to 500% of the outer diameter of the outer tubular body and more preferably, 100% to 400% of the outer diameter of the outer tubular body. In general, the flow lumen created by the leaflets will taper from a larger shape/area adjacent the prosthesis inlet to a smaller shape/area adjacent the prosthesis outlet. The transition from larger to smaller shape/area can be linear over the length of the leaflets or the rate of change can increase or decrease along the length of the leaflet depending on the desired performance characteristics to be achieved.

In a preferred embodiment, the outlet ends of the leaflets will have a “normally open” configuration to allow minimally restricted antegrade blood flow (in a direction from the inlet to the outlet of the prosthesis). The inlet ends of the apposed leaflets are typically shaped symmetrically to each other (with a plane of symmetry defined by the longitudinal and major axes of the outer tubular body as shown in FIG. 1A) to further minimize restriction to blood flow in the antegrade direction. Each leaflet will be arranged within the outer tubular body with a width or lateral dimension generally parallel to the major axis as shown in FIG. 1A and a thickness parallel to the minor axis as shown in FIG. 1A. A length of each leaflet extends along a longitudinal axis of the outer tubular body as shown in FIG. 1A.

In a preferred embodiment, lateral edges of the leaflets will be attached to or integrated with the inner wall of the outer tubular body in the longitudinal direction. The inlet end of each leaflet will typically follow a curve or arc between the lateral edges and will also be attached to or integrated with the inner surface of the outer tubular body proximate the inlet end thereof. In contrast, the outlet ends and opposed surfaces of each leaflet will be free from attachment to the outer tubular body, allowing the outlet ends to freely open and close in response to reversing blood flow through the lumen of the outer tubular body.

Attachment or integration of the leaflets along their lateral edges and inlet ends improves the columnar strength of the leaflets to resist leaflet collapse and/or leaflet inversion when the valve leaflets coapt in response to retrograde blood flow. Separation of leaflets along their outlet edges allows the leaflets to coapt under retrograde flow. The leaflet thickness can be uniform around the periphery or can vary to achieve different performance characteristics, such as, but not limited to, reducing the thickness at the minor aspect to improve leaflet coaptation.

The shape of the outlet ends of the leaflets will usually be non-linear when viewed in a direction parallel to the minor axis of the outer tubular body, as shown in FIG. 1A. When linear or projected to be linear, the length of the outlet edge of each leaflet will typically be from 75% to 150% of the width of the inner tubular body, usually being from 95% to 125% of the width of the inner tubular body, when measured along the major axis as shown in FIG. 1A. An outlet edge length in this range allows the leaflets to symmetrically form a linear or near-linear line of coaptation/seal under retrograde flow when viewed along the longitudinal axis of the outer tubular body (i.e. looking through the lumen of the outer tubular body from the outlet end toward the inlet end). When the outlet edge is linear or projected to be linear, larger outlet opening areas will result in longer outlet leaflet edge lengths, which will increase the tendency of the coaptation/seal line to be nonlinear (wrinkle) and thus will be generally less preferred.

In a preferred embodiment, the outlet end of each leaflet is configured to increase the open cross-sectional area between the leaflets when they are open to minimize the magnitude and time of velocity increase as the blood flows through the open valve in the antegrade direction. Typically, the outlet edge of each leaflet is recessed in a direction toward the inlet end of the valve leaflet. In specific embodiments, the leaflets will be recessed in an arc, such as a generally parabolic, ellipsoidal, or other smooth arc, when viewed in a direction along a minor axis of the outer tubular member. V-shaped and other recessed configurations may also find use. In exemplary embodiments, an open area is present between the outlet ends of the valve leaflets when viewed through the prosthesis lumen in a direction along the longitudinal axis of the outer tubular body. The open area is preferably at least 20% of the valve prosthesis inlet cross-sectional area, more preferably being at least 30%, and sometimes at least 40%, typically being in a range between 20% to 80% of the valve prosthesis inlet cross-sectional area, more typically being between 30% to 70%.

The valve leaflets and deformable body will typically be elastic or deformable, usually having a tensile strength in a range from 1.3 MPa to 15 MPa, typically from 4 MPa to 10 MPa. The outlet ends of valve leaflet remain open so long as the leaflets are free from stress. As blood flows from the inlet end to the outlet end of the valve, the blood flow will apply force over inlet surfaces of the leaflets, causing the leaflets to stretch, elongate, or dilate and further open the valve. When the blood flow reverses, a force will be applied to the outlet surfaces of the leaflets, distending, elongating, or stretching the leaflets and causing the outlet ends to close and seal in a linear or near linear line of coaptation. The leaflets will remain closed for so long as the blood pressure on the outlet sides of the leaflets exceeds the blood pressure on the inlet sides of the leaflets.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

The invention will be understood from the following description of preferred embodiments, taken in conjunction with the accompanying drawings, wherein:

FIG. 1A is perspective view defining the axis orientation for an exemplary valve prosthesis;

FIG. 1B is a perspective view of one embodiment of an exemplary valve prosthesis having an outer tubular body and a valve with leaflets having a recessed (nonlinear) terminus shape;

FIG. 2 is a cross-sectional, perspective view taken along a longitudinal axis of the exemplary valve prosthesis of FIG. 1;

FIG. 3 is a cross-sectional, side view taken along a longitudinal axis of the exemplary valve prosthesis of FIG. 1;

FIG. 4A is another perspective of the prosthesis FIG. 1, depicting the valve inside of the outer tubular body with hidden lines;

FIG. 4B is a perspective of the valve of the prosthesis FIG. 1, with the outer tubular body removed;

FIG. 5 is a perspective view of another embodiment of the prosthesis with outer tubular body of the present invention having a valve with a leaflet having a projected linear terminus shape, wherein the apposed leaflets are opened;

FIG. 6 is an end view of the valve of FIG. 5 illustrating the outlet area where the terminus shape of the leaflets is linear or projected to be linear and normally open;

FIG. 7 is end view of the valve of FIG. 5, with the outer tubular body, illustrating the outlet area where the terminus shape of the leaflets is nonlinear and normally open;

FIG. 8 is a perspective view of a valve prosthesis depicting an encapsulated reinforcement or other structural element(s) within the wall of the outer tubular body;

FIG. 9 is another perspective view of the valve prosthesis from FIG. 8 depicting a partially encapsulated reinforcement or other structural element(s), incorporate features to provide stability and/or migration resistance, within the wall of the outer tubular body;

FIG. 10 is a perspective cross-sectional view of the valve prosthesis taken along a longitudinal axis from FIG. 9 depicting a deployed position within a vessel with normally open valve leaflets and antegrade flow;

FIG. 11 is another perspective cross-sectional view of the valve prosthesis taken along a longitudinal axis from FIG. 10 depicting a deployed position within a vessel with the valve leaflets closed due to retrograde flow.

### DETAILED DESCRIPTION OF THE INVENTION

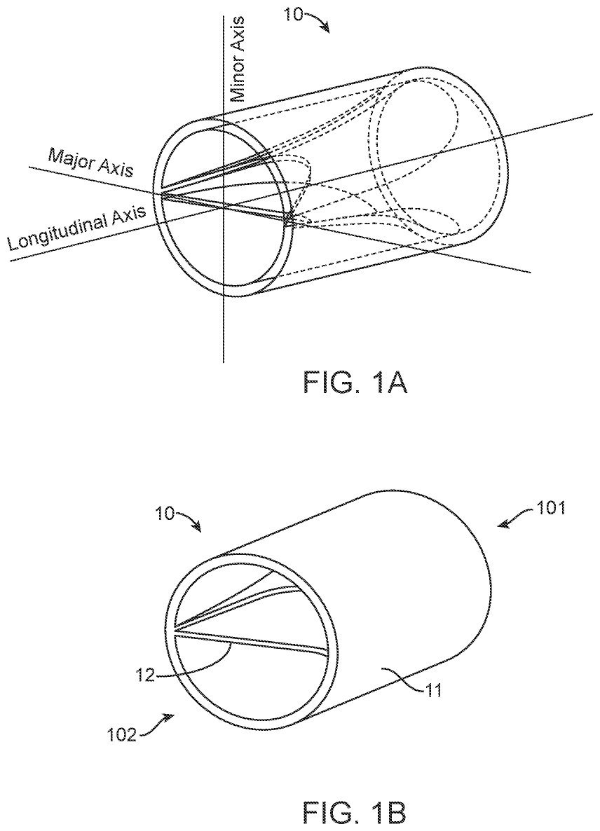

FIG. 1A defines the axis orientation of the valve prosthesis 10 of the present invention. The major axis is aligned with the longest aspect of the leaflet terminus shape and the minor axis is aligned with the shortest aspect of the leaflet terminus shape. The longitudinal axis is parallel to the valve prosthesis flow lumen.

FIG. 1B depicts one embodiment of a valve prosthesis 10 constructed in accordance with the principles of the present invention. The valve prosthesis 10 typically comprises an outer tubular body 11, typically defining a tubular wall or body, and a valve 12. The outer tubular body 11 has an inlet end 101 and an outlet end 102, where the valve prosthesis is implanted in a vein or other blood vessel so that blood normally flows into the inlet end and out of the outflow end, The valve 12 is disposed to close in the presence of retrograde flow (i.e. in a direction from outlet to inlet) where retrograde blood flow causes a pair of apposed leaflets to close against each other to shut off such retrograde flow. Thus, the valve prosthesis 10, when implanted in a vein or other blood vessel, will allow flow in the normal direction (for example back toward the heart in the venous system) while blocking flow in the retrograde direction.

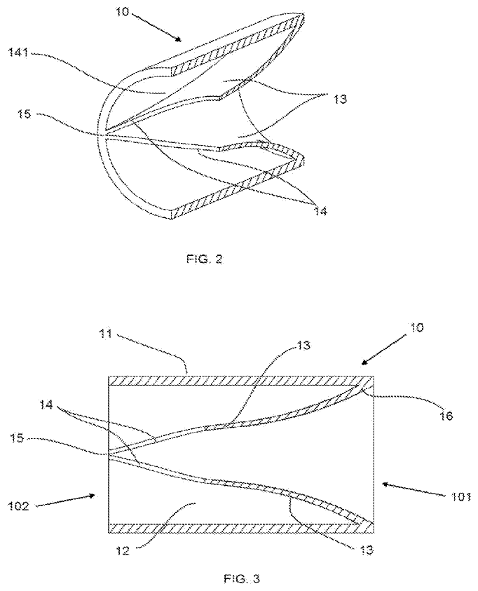

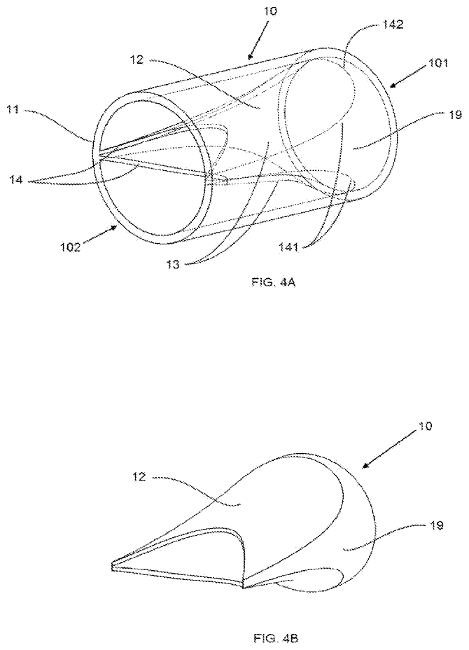

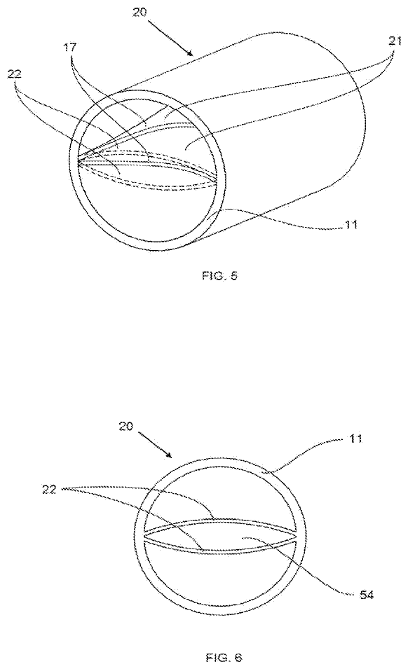

FIG. 2 is an axial, cross-sectional view of the valve prosthesis 10 depicting two valve leaflets 13, each having recessed, typically arcuate or curved, outlet edges (terminus shape), which are nonlinear when viewed perpendicular to the prosthesis lumen and aligned with the minor axis of the leaflets 14, and which are normally open. The leaflets 13 each have lateral edges 141 which terminate at location 15 adjacent the outlet end of the valve prosthesis 10 and diverge in a direction toward the inlet end, as best seen in FIG. 4A. Typically, the lateral edges will be integrated into the wall of the outer tubular body, usually being molded or otherwise fabricated from the same material to form an integrated structure comprising the outer tubular body and the leaflets.

FIG. 3 is a side, axial cross-sectional view of the valve prosthesis 10 depicting an attachment location 16 of the valve leaflets 13 adjacent the inlet end 101 of the valve prosthesis. The leaflets 13 are shown to taper along their lengths, typically from a thicker section near the inlet end 101 of the valve prosthesis 10 to a thinner section nearer the outlet end 102.

FIG. 4A is another perspective view of the prosthesis 10 with the valve 12 shown with hidden lines within the outer tubular body 11. The outer tubular body 11 and the valve 12 share a “connection surface 19” to form an integrated or homogeneous structure, between the valve 12 and the outer tubular body 11. In particular, it can be seen that the lateral edges 141 of each valve leaflet 13 transition into an inlet edge 142 which is joined or integrated to the inlet end 101 of the outer tubular body.

FIG. 4B is another perspective view of the prosthetic valve 12 from FIG. 4A with the outer tubular body removed that depicts the connection or shared surface 19 of the valve 12 and outer tubular body 11.

FIG. 5 depicts a valve prosthesis 20 with an outer tubular body 11 having leaflets 21 with recessed outlet edges 17 shown in their open configuration as they would appear in the presence of antegrade blood flow from the inlet end to the outlet end of the valve. The leaflets 21 are normally open to allow antegrade blood flow with limited restriction. The benefit of the recess can be seen by comparison with the much smaller opening which would be present if the leaflets were not recessed as shown by broken lines 22.

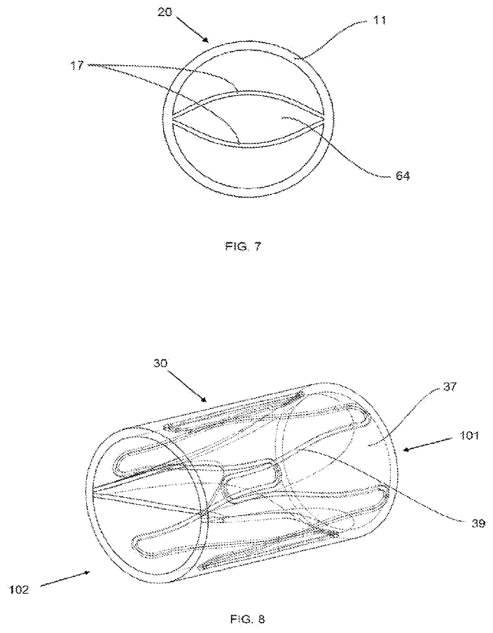

FIG. 6 is an end view of the outer tubular body 11 of the valve prosthesis 20 of FIG. 5 showing the relatively limited area 54 provided by straight outlet edges 22 (broken line in FIG. 5).

FIG. 7 is an end view of the outer tubular body 11 of the valve prosthesis 20 of FIG. 5 showing the relatively larger area 64 provided by recessed outlet edges 17.

FIG. 8 is a perspective view of another embodiment of valve prosthesis 30 with an encapsulated reinforcement or other structural element(s) 39, shown with hidden lines, within the wall of the outer tubular body 37. The outer tubular body inlet end 101 and an outlet end 102 are also shown. The encapsulated reinforcement or other structural element(s) 39 are preferably in the form of a scaffold structure similar to a self-expanding or balloon-expandable stent structure. The scaffold will usually be embedded wholly or partially within the outer tubular body 37, but alternatively may be coupled to all or a portion of an outer or inner surface of the outer tubular body 37.

FIG. 9 is a perspective view of the valve prosthesis 30 from FIG. 8 where the encapsulated reinforcement or other structural element(s) 39, shown with hidden lines, includes barb or hook features 40 to provide stability and/or migration resistance when implanted/deployed within a vessel. The base or matrix material of the outer tubular body 37, which encapsulates the reinforcement or other structural element(s) 39, is shown in this embodiment not covering/encapsulating the hook or barb features 40. The hook and barb features could also include a sharpened tip/point 41 to enhance the ability to penetrate into the native vessel during or after deployment.

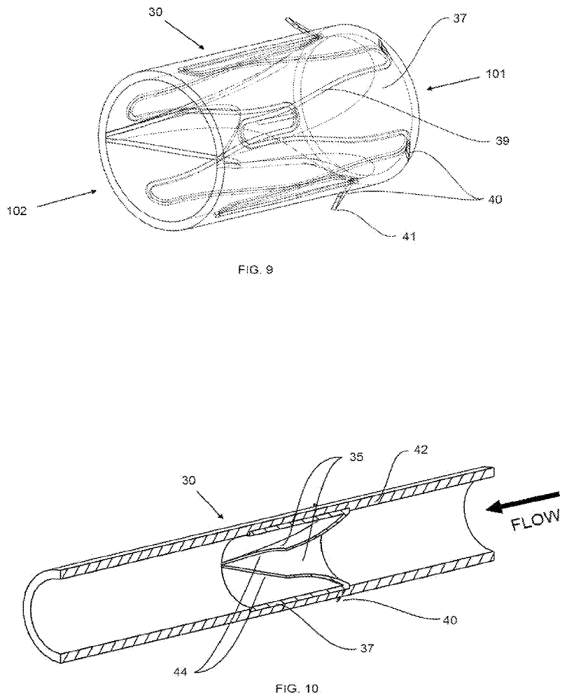

FIG. 10 is a perspective cross-sectional view of the valve prosthesis 30 from FIG. 9 shown deployed/implanted in the lumen of a native vessel 42. In this depiction, the outer tubular body 37 with encapsulated reinforcement or other structural element(s) that incorporates hook and barb features 40 have penetrated into the wall of the native vessel. The normally open valve leaflets 35 with nonlinear, curved and recessed outlet edges 44, when viewed perpendicular to the prosthesis lumen and aligned with the minor axis of the leaflets, allow the antegrade flow to pass through the valve with limited or no resistance.

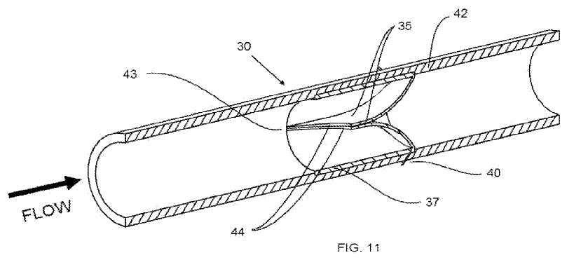

FIG. 11 is a perspective cross-sectional view of the valve prosthesis 30 from FIG. 10 where the normally open valves leaflets 35 with nonlinear, curved and recessed outlet edges 44, when viewed perpendicular to the prosthesis lumen and aligned with the minor axis of the leaflets, form a linear or near linear coaptation 43 of outlet edges 44 during retrograde blood flow.

As can be seen in FIG. 11, the outlet edges 44 of the leaflets 35 will close or coapt over a distance sufficient to provide a stable seal and prevent retrograde blood flow for so long as the “downstream” pressure exceeds the “upstream” pressure. The elasticity of the individual leaflets allows stretching, elongation, or distention of the leaflets (which are normally open when unstressed and which must stretch/distend to close) contributing significantly to this ability to provide a reliable seal.

It will be appreciated that the components and/or features of preferred embodiments described herein may be used together or separately. The preferred embodiments of the invention are described above in detail for the purpose of setting forth a complete disclosure and for the sake of explanation and clarity. Those skilled in the art will envision other modifications within the scope and sprit of the present disclosure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An implantable prosthetic valve comprising:
an outer tubular body that having an axial passage extending from an inlet end to an outlet end, the body being constructed of a flexible biocompatible material; and
a pair of apposed valve leaflets disposed in the axial passageway of the outer tubular body.
wherein each valve leaflet has an inlet edge attached along its entire length to an inner wall of the outer tubular body and an outlet edge attached only at opposed lateral ends to the inner wall of the outer tubular so that inlet edges open or remain open when fluid pressure is applied to the inlet end and close together when fluid pressure is applied to the outlet end; and
wherein the outlet edge of each leaflet is recessed along its length in a direction toward the inlet end.

2. The valve of claim 1, wherein the recessed outlet edges are curved away in a direction toward the inlet end.

3. The valve of claim 1, wherein the valve leaflets are joined to an inner wall of the outer tubular body.

4. The valve of claim 3, wherein the valve leaflets have lateral edges that follow converging paths along the inner wall from their inlet ends to their outlet ends.

5. The valve of claim 1, wherein the outer tubular body and the valve leaflets are formed from a biocompatible polymer.

6. The valve of claim 5, wherein the outer tubular body and the valve leaflets are integrally formed from the biocompatible polymer.

7. The valve of claim 6, wherein the outer tubular body and the valve leaflets are integrally formed by molding or welding.

8. The valve of claim 1, wherein the outer tubular body is reinforced.

9. The valve of claim 8, further comprising a reinforcement scaffold coupled to the outer tubular body.

10. The valve of claim 9, wherein the scaffold is embedded within the outer tubular body.

11. The valve of claim 9, wherein the scaffold is self-expanding.

12. The valve of claim 9, wherein the scaffold is balloon expandable.

13. An implantable prosthetic valve comprising:
an outer tubular wall having an axial passage extending from an inlet end to an outlet end, the wall being constructed of a flexible biocompatible material; and
a pair of apposed valve leaflets disposed in the axial passageway of the outer tubular body;
wherein each valve leaflet has an inlet edge, an outlet edge, and a pair of lateral edges extending between the inlet edge and the outlet edge;
wherein the inlet edge, the outlet edge, and the pair of lateral edges of each leaflet are integrated along their entire lengths into the outer tubular wall; and
wherein the outlet edges are configured to open or remain open when fluid pressure is applied to the inlet end and close together when fluid pressure is applied to the outlet end.

14. The valve of claim 13, wherein the outlet edge of each leaflet is recessed along its length in a direction toward the inlet end.

15. The valve of claim 13, wherein the outlet edge of each leaflet is straight along its length in a lateral direction.

16. The valve of claim 13, wherein the outer tubular wall and the valve leaflets are formed from a biocompatible polymer.

17. The valve of claim 13, wherein the outer tubular wall is reinforced.

18. The valve of claim 13, further comprising a reinforcement stent in the tubular wall.

19. The valve of claim 18, wherein the scaffold is embedded within the tubular wall.

20. The valve of claim 13, wherein the scaffold is self-expanding.

21. The valve of claim 13, wherein the scaffold is balloon expandable.
